(19) United States
(12) Patent Application Publication
(10) Pub. No.: US 2003/0204268 A1
(43) Pub. Date: Oct. 30, 2003

(54) Binary attachment mechanism and method for a modular prosthesis

(75) Inventors: Daniel E. Gerbec, Logan, UT (US); T. Wade Fallin, Hyde Part, UT (US)

Correspondence Address: DANA L. TANGREN, WORKMAN NYDEGGER & SEELEY, P.O. BOX 45862, SALT LAKE CITY, UT, 84145, US

(73) Assignee: MEDICINELODGE, INC.

(21) Appl. No.: 10/132,668

(22) Filed: Apr. 25, 2002

(51) Int. Cl. A61F 2/30

(52) U.S. Cl. 623/23.44; 623/22.42; 623/20.15; 623/911

(57) ABSTRACT

A binary attachment mechanism for a modular prosthesis comprises a body and a stem. The body has a top surface, a bottom surface, an internal surface bounding a bore extending between the top and bottom surface. The stem has a protrusion having an external surface adapted to be received in the bore of the body. Sliding the protrusion into the bore causes the external surface of the protrusion to form discrete, spaced apart, releasable connections with the internal surface of the body.

### CROSS-REFERENCE TO RELATED APPLICATIONS

Not applicable.

### BACKGROUND OF THE INVENTION

1. The Field of the Invention

The present invention relates generally to modular orthopedic prostheses and, more specifically, to attachment mechanisms for securing components of a modular orthopedic prosthesis.

2. The Relevant Technology

Modular orthopedic prostheses offer many advantages to the user. By selecting independent modular components to construct a complete prosthesis, custom fitting of a patient's specific anatomy or specific bony condition can be accomplished.

Several attachment mechanisms are known in the art for connecting the components of a modular prosthesis. Generally, any two modular components are connected by one contiguous interface. Even three-piece modular connections typically rely on only one contiguous connection interface between any two modular components.

Because of the high physiological loads borne by the skeletal structure, orthopedic prostheses are subject to high bending, shear, and torsional loads. Where a single contiguous connection is used to connect components of a modular prosthesis, the applied loads can be localized, thereby increasing the failure at that point. It would therefore be an improvement in the art to provide modular orthopedic prostheses that can better withstand the mechanical service loads by better distributing the loads acting upon the prosthesis.

Furthermore, one of the advantages of modular orthopedic prostheses is the capacity to select, at the time of surgery, a desired orientation between modular components. Many modular connections known in the art do not facilitate a state of partial assembly that closely replicates the final longitudinal configuration of the prosthesis, where, in the state of partial assembly, the modular components can be freely rotated with respect to each other. It would therefore be another improvement in the art to provide modular prostheses that would accommodate a state of partial assembly that closely replicates the longitudinal configuration of the prosthesis while permitting free relative rotation between the modular components.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1A is a cross sectional view of a binary attachment mechanism in a disassembled state.

FIG. 1B is the binary attachment mechanism shown in FIG. 1A in a partially assembled state.

FIG. 1C is the binary attachment mechanism shown in FIG. 1A in a fully assembled state.

FIG. 2 is a cross sectional view of an alternate embodiment of an assembled binary attachment mechanism.

FIG. 3 is a cross sectional view of another alternate embodiment of an assembled binary attachment mechanism in a disassembled state.

FIG. 4 is a cross sectional view of yet another alternate embodiment of an assembled binary attachment mechanism.

FIG. 5A is a cross sectional view of still another alternate embodiment of a binary attachment mechanism in a partially assembled state.

FIG. 5B is the binary attachment mechanism shown in FIG. 5A in a fully assembled state.

FIG. 6 is a cross sectional view of a modular hip implant having components connected together by a binary attachment mechanism.

FIG. 7 is a cross sectional view of a modular tibial knee implant having components connected together by a binary attachment mechanism.

FIG. 8 is a cross sectional view of a modular intramedullary rod having components connected together by a binary attachment mechanism.

### DETAILED DESCRIPTION OF THE PREFERRED EMBODIMENTS

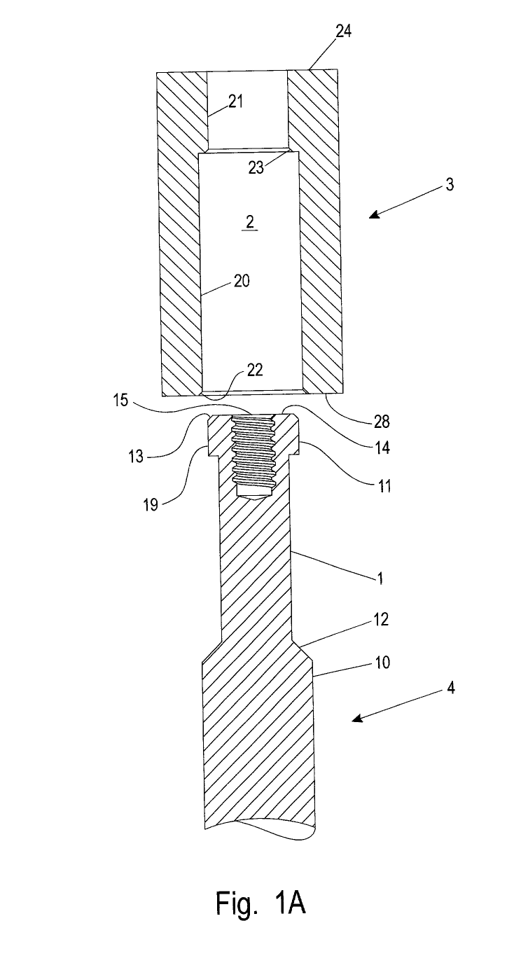

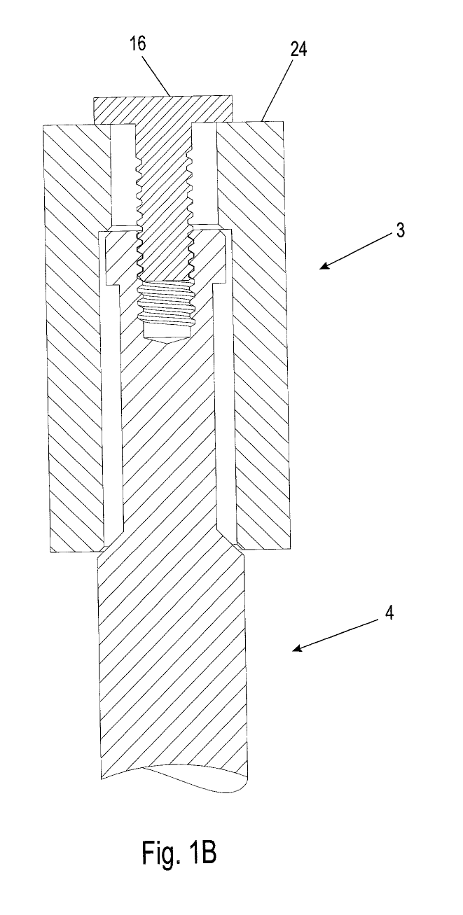

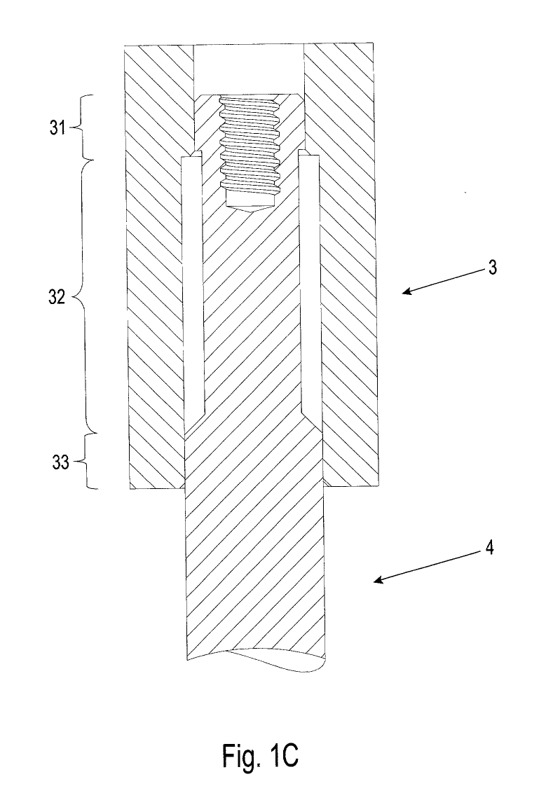

Referring to one or more of the preferred embodiments of the present invention as depicted in FIGS. 1-8, there are two components, a body 3 and a stem 4, adapted to connect to each other to form a binary, or two-piece, modular prosthesis assembly. Body 3 and stem 4 may be made from any suitable biocompatible material that can withstand the physiological loads during the lifetime of the implant. Preferentially, body 3 and stem 4 would be made from biocompatible metals, such as titanium alloys, zirconium alloys, cobalt chromium alloys, and stainless steels.

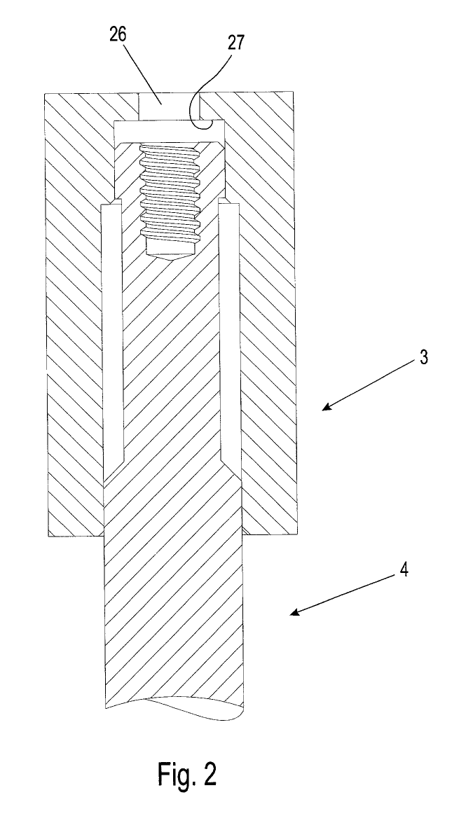

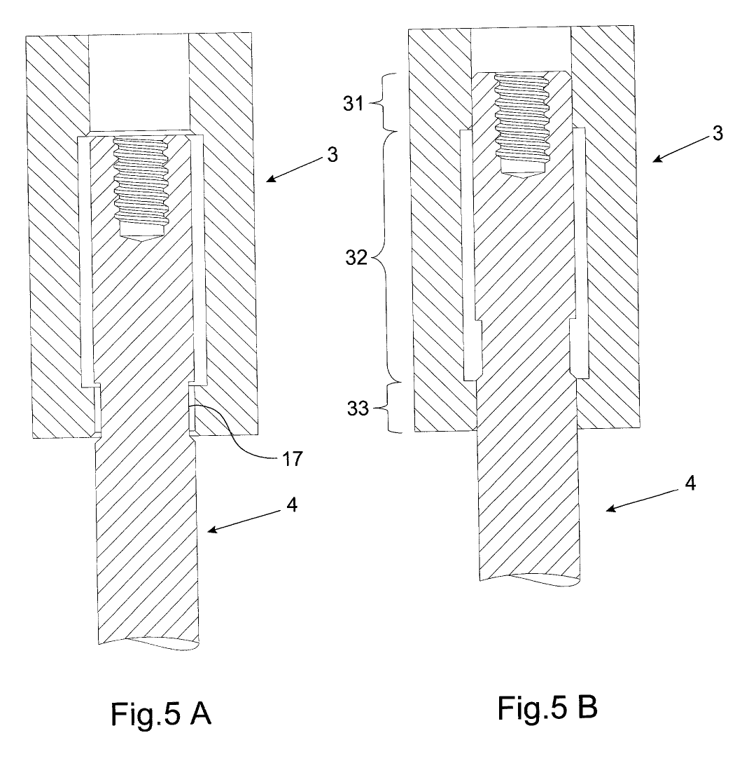

Body 3 has a bore 2 bounded by an internal surface extending between a top end 24 and a bottom end 28. The internal surface of bore 2 has an upper socket wall 21 extending from top end 24 to a transition surface 23. The internal surface of bore 2 further has a lower socket wall 20 extending from transition surface 23 to bottom end 28. Alternatively, lower socket wall 20 may extend from upper transition surface 23 to a lower transition surface 22 as shown in FIGS. 2-5. In the preferred embodiment, socket wall 21 defines a diameter that is smaller than a diameter defined by socket wall 20 as shown in FIGS. 1-4. Alternatively, the diameter of socket wall 21 is the same as the diameter of socket wall 20 as depicted in FIG. 5. Additionally, bore 2 may include an access hole 26 extending from top end 24 to a shoulder 27 and, correspondingly, upper socket 21 may extend from the shoulder 27 to the upper transition surface 23 as depicted in FIGS. 2 and 7.

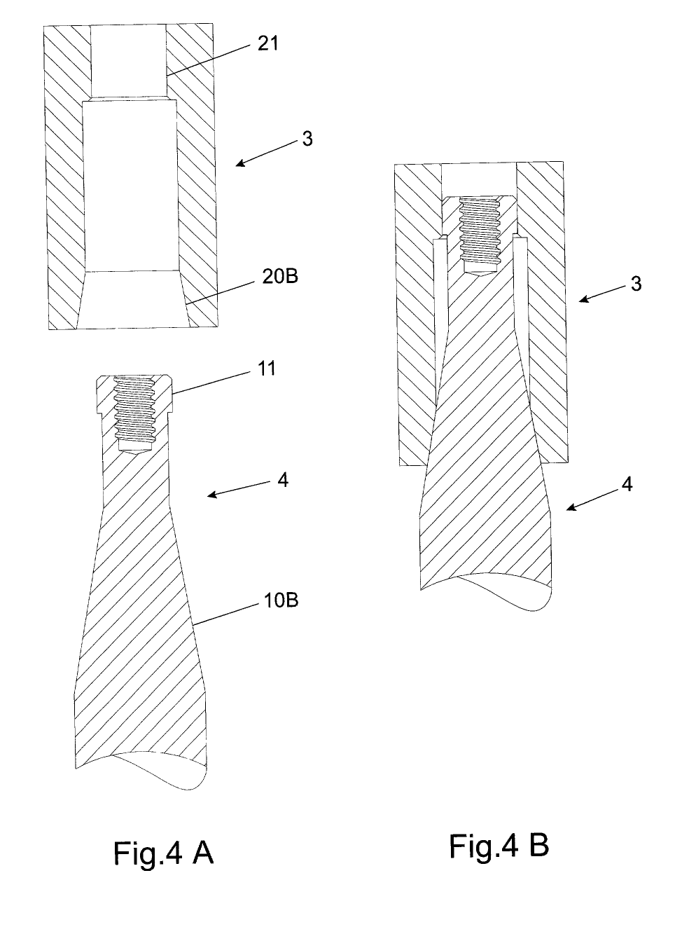

The upper and lower transition surfaces, 23 and 22, help guide protrusion 1 into bore 2. Transition surfaces 23 and 22 can be in the form of an internal chamfer as depicted in FIGS. 2 and 4, or in the form of a shoulder as depicted in FIGS. 1 and 5.

Stem 4 has a protrusion 1 which is the upper end of stem 4, and protrusion 1 is adapted to slide into the bore 2. Protrusion 1 has a free end 14 and an external surface 19 descending longitudinally downward from free end 14. The external surface 19 is comprised of upper surface 11 and lower surface 10. Alternatively, the external surface 19 of protrusion 1 may include upper transition surface 13 and lower transition surface 12 as depicted in FIG. 1. Furthermore, protrusion 1 includes a female thread 15 extending down from free end 14 to facilitate assembly of body 3 to stem 4.

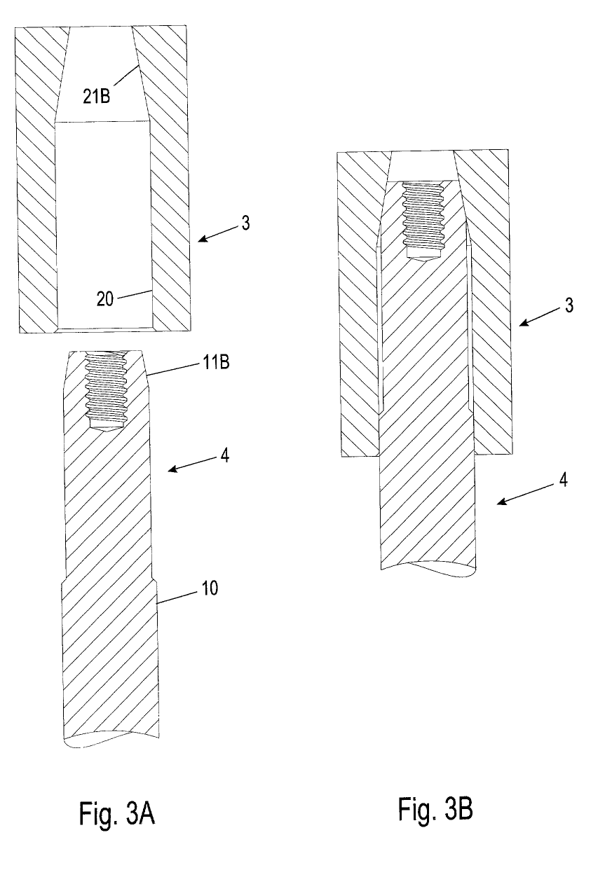

The upper and lower transition surfaces, 13 and 12, help guide protrusion 1 into bore 2. Transition surfaces 13 and 12 can be in the form of an internal chamfer as depicted in FIG. 1, or in the form of a shoulder as depicted in FIG. 3.

To assemble the stem 4 to the body 3, protrusion 1 is slid partially into the bore 2 as depicted in FIG. 1B. As depicted in FIGS. 1-4, upper surface 11 is sized to slide freely past lower socket wall 20. With the components 3 and 4 partially assembled, upper surface 11 acts like a trunnion constrained by lower socket wall 20 to define an axis of rotation, permitting the body 3 and the stem 4 to be placed into a desired rotational orientation with respect to each other before final assembly. A threaded fastener 16 is provided as a tool to draw the stem 4 towards the body 3, thereby drawing the protrusion 1 into the bore 2 to cause the upper surface 11 and lower surface 10 to form simultaneous, discrete, and releasable connections with the upper socket wall 21 and lower socket wall 20, respectively. The upper surface 11 and upper socket wall 21 define a first connection length 31, and the lower surface 10 and the lower socket wall 20 define a second connection length 33. Connection length 31 and connection length 33 are spaced apart by distance 32.

The releasable connections may be in the form of a press fit or a self-locking taper. Both the press fit and the self-locking taper provide for frictional biasing between the external surface 19 of the stem and the internal surface of the body. The frictional biasing provides a releasable connection that relies on a recoverable elastic deformation of the mating internal and external surfaces.

In one embodiment the distance 32 between the releasable connections is generally greater than sum of the connection lengths 31 and 33, and preferably the distance between the releasable connections is at least greater than the shortest of the connection lengths 31 and 33. Other distances can also be used. For example, the distance 32 between the connections can be in a range between about 5 mm to about 50 mm or can simply be larger than 5 mm, 10 mm, or 15 mm. By increasing the distance 32 between the connections, reaction forces and stresses associated with the connections are decreased when bending loads act upon the assembled body 3 and stem 4. Decreased reaction forces and stresses provide for higher performance assemblies that can carry higher bending loads and reduce fretting caused by cyclic loads. Furthermore, the higher performance assembly can enable smaller sizes that sufficiently withstand physiological loads.

To enable releasable press fit connections in one embodiment, the amount of interference between the surfaces 10 and 11 and the socket walls 20 and 21, respectively, is less than the radial yield strain of the chosen material, and preferably less than 75% of the radial yield strain. To ensure that a press fit is achieved, the interference between the surfaces 10 and 11 and the socket walls 20 and 21, respectively, is typically at least 10% of the radial yield strain and preferably greater than 25% of the radial yield strain. For example, provided that the upper surface 11 of stem 4 defines a diameter of 0.500 inch, and provided that the stem 4 and body 3 are made from titanium alloy with 6% vanadium and 4% aluminum, then the yield strain would be approximately 0.0035 inch. Therefore, the preferred interference would be greater than 0.0009 inch and less than 0.0027 inch.

The connection lengths 31 and 32 should be of sufficient length to produce a connection strength that can withstand physiological loads, yet the connection lengths 31 and 32 must remain short enough to that assembly loads are not excessive. In one embodiment the connection length is in a range between about 0.020 inch and 0.500 inch, and preferably between about 0.040 inch and about 0.100 inch, although other ranges can also be used.

A self-locking taper may be used in combination with a press fit to form the releasable connections. The self-locking taper may be present at the upper surface 11B and upper socket 21B as depicted in FIG. 3A, or the self-locking taper may be present at the lower surface 10B and lower socket 20B as depicted in FIG. 4A. Generally speaking, the self-locking taper would have an included angle between 2° and 8°, and preferably the self-locking taper would have an included angle between 3° and 6°. Other angles can also be used.

An alternate embodiment of the present invention is depicted in FIGS. 5A and 5B. The protrusion includes an undercut 17 positioned between the upper surface 11 and the lower surface 10. Furthermore, upper surface 11 and lower surface 10 are nominally the same size, and, correspondingly, upper socket wall 21 and lower socket wall 22 are nominally the same size. Where both connections are in the form of a press fit, and where the interference associated with the press fit is nominally the same for both connections, then a certain force would be required to move upper surface 11 to a position above lower socket wall 22. When upper surface 11 is located above lower socket wall 20 and below upper socket wall 11, undercut 17 is adapted to provide clearance around lower socket wall 20. In this arrangement, stem 4 is prevented from inadvertently moving out of body 3, yet stem 4 is free to rotate with respect to body 3, thereby allowing the user to create a desired rotation between body 3 and stem 4. Once the desired rotation is achieved, body 3 can be assembled to stem 4 in the manner previously described.

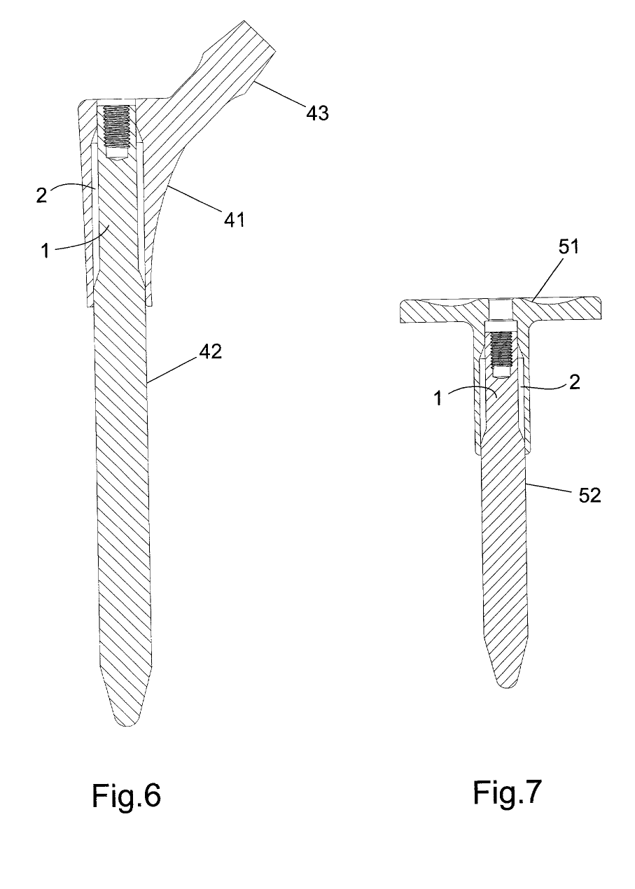

Depicted in FIG. 6 is a modular femoral hip implant, wherein a neck 41 is analogous to the body 3 shown in FIGS. 1-5, and a stem 42 is analogous to the stem 4 shown in FIGS. 1-5. The neck 41 is designed to fit into a proximal femur that has a resected femoral head. The stem 42 is designed to fit into the intramedullary canal of the femur. The neck 41 has bore 2 and the stem 42 has protrusion 1. Frustoconical surface 43 is adapted to carry a spherical ball (not shown) adapted to articulate with a prosthetic or natural acetabulum (not shown). It is appreciated that any of the embodiments depicted in FIGS. 1-5 can be substituted to permit secure attachment between neck 41 and stem 42.

Depicted in FIG. 7 is a modular tibial knee implant, wherein a plate 51 is analogous to the body 3 shown in FIGS. 1-5, and a stem 52 is analogous to the stem 4 shown in FIGS. 1-5. The plate 51 is designed to fit onto a proximal tibia that has its upper most surface resected. The stem 52 is designed to fit into the intramedullary canal of the tibia. The plate 51 has bore 2 and the stem 52 has protrusion 1. It is appreciated that any of the embodiments depicted in FIGS. 1-5 can be substituted to permit secure attachment between plate 51 and stem 52.

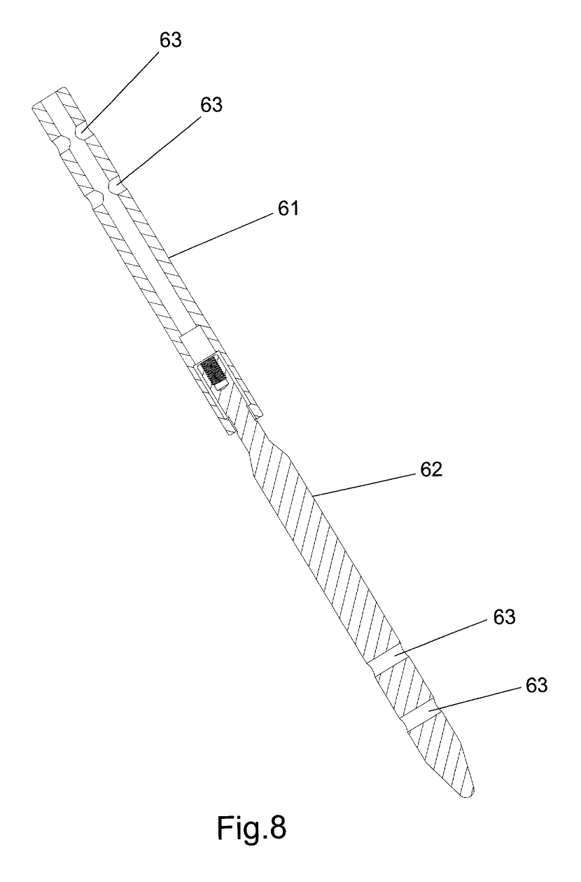

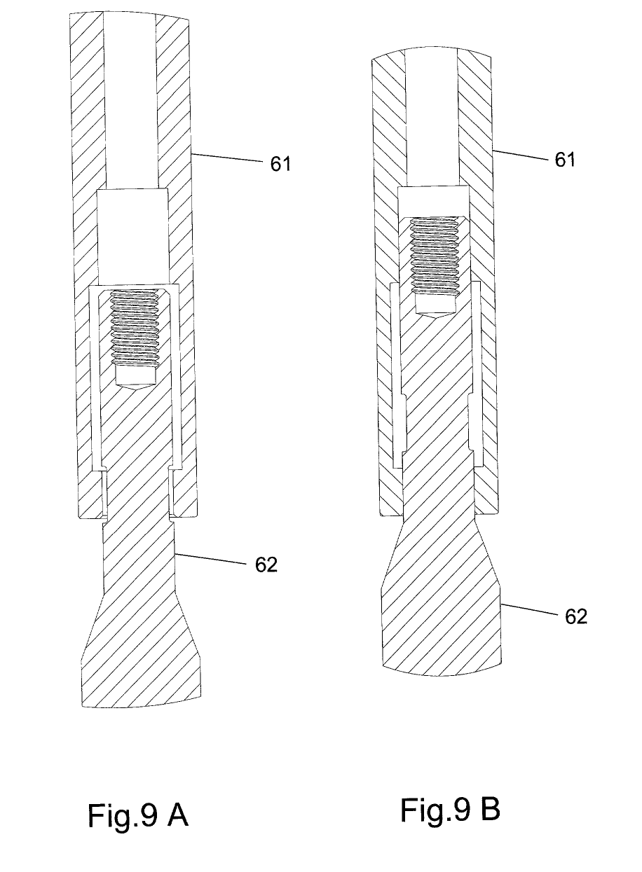

Depicted in FIG. 8 is a modular intramedullary rod for stabilizing fractures of long bones. The proximal module 61 is analogous to the body 3 shown in FIGS. 1-5, and a distal module 62 is analogous to the stem 4 shown in FIGS. 1-5. The proximal module 61 and distal module 62 are designed to fit within the intramedullary canal of a long bone, such as a femur, tibia, or humerus. Both the proximal module 61 and the distal module 62 have holes to accommodate interlocking bone screws. If desired, the relative rotational position between the holes 63 in the proximal and distal modules 61 and 62 can be selected at the time of surgery to better align with bone fragments. It is appreciated that any of the embodiments depicted in FIGS. 1-5 can be substituted to permit secure attachment between proximal module 61 and distal module 62.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An attachment mechanism for securely connecting components of a modular prosthesis, the attachment mechanism comprising:
a stem having a protrusion, the protrusion having an external surface descending longitudinally downward from a free end, the external surface comprising an upper surface and a longitudinally spaced apart lower surface; and
a body having an internal surface bounding a bore, the internal surface comprising an upper socket wall and a longitudinally spaced apart lower socket wall;
whereby sliding the protrusion into the bore causes the upper and lower surfaces to form, discrete and releasable connections with the upper and lower socket walls, respectively, and wherein each connection defines a connection length and the connections are spaced apart by a distance greater than at least one of the connection lengths.

2. The attachment mechanism of claim 1, wherein at least one of the releasable connections is a press fit.

3. The attachment mechanism of claim 1, wherein at least one of the releasable connections is a self-locking taper.

4. The attachment mechanism of claim 1, wherein one releasable connection is a press fit and the other releasable connection is a self-locking taper.

5. The attachment mechanism of claim 1, wherein the upper surface is contiguous.

6. The attachment mechanism of claim 2, wherein the surfaces and the socket walls each define a radial yield strain, wherein the press fit is generally between 10% and 90% of the lowest radial yield strain.

7. The attachment mechanism of claim 2, wherein the surfaces and the socket walls each define a radial yield strain, wherein the press fit is generally between 25% and 75% of the lowest radial yield strain.

8. The attachment mechanism of claim 2, wherein each of the connection lengths is generally between 0.020 inch and 0.500 inch.

9. The attachment mechanism of claim 2, wherein each of the connection lengths is generally between 0.040 inch and 0.100 inch.

10. The attachment mechanism of claim 1, wherein the body has a proximal end with the upper socket wall formed thereat and an opposing distal end with lower socket wall formed thereat.

11. The attachment mechanism of claim 1, wherein the connections are separated by at least 5 mm.

12. An attachment mechanism for securely connecting components of a modular prosthesis, the attachment mechanism comprising:
a stem having a protrusion, the protrusion having an external surface descending longitudinally downward from a free end, the external surface comprising an upper surface and a longitudinally spaced apart lower surface, each surface defining a diameter, the upper surface diameter being smaller than the lower surface diameter; and
a body having an internal surface bounding a bore, the internal surface comprising an upper socket wall and a longitudinally spaced apart lower socket wall, each socket wall defining a diameter, the upper socket wall diameter being smaller than the lower socket wall diameter;
whereby sliding the protrusion into the bore causes the upper and lower surfaces to form discrete releasable connections with the upper and lower socket walls, respectively and wherein each connection defines a connection length and the connections are spaced apart by a distance greater than at least one of the connection lengths.

13. The attachment mechanism of claim 12, wherein at least one of the releasable connections is a press fit.

14. The attachment mechanism of claim 12, wherein at least one of the releasable connections is a self-locking taper.

15. The attachment mechanism of claims 12, wherein one releasable connection is a press fit and the other releasable connection is a self-locking taper.

16. The attachment mechanism of claim 12, wherein the protrusion further includes a tapered surface interposed between the upper and lower surfaces.

17. The attachment mechanism of claim 12, wherein the internal surface further includes a tapered surface interposed between the upper and lower socket walls.

18. The attachment mechanism of claim 12, wherein the protrusion further includes a tapered surface located above the upper surface.

19. The attachment mechanism of claim 12, wherein the internal surface further includes a tapered surface located below the lower socket wall.

20. An attachment mechanism for securely connecting components of a modular prosthesis, the attachment mechanism comprising:
a stem having a protrusion, the protrusion having an external surface descending longitudinally downward from a free end, the external surface comprising an upper surface and a longitudinally spaced apart lower surface, each surface defining a diameter, the upper surface diameter being substantially the same as the lower surface diameter; and
a body having an internal surface bounding a bore, the internal surface comprising an upper socket wall and a longitudinally spaced apart lower socket wall, each socket wall defining a diameter, the upper socket wall diameter being substantially the same as the lower socket wall diameter;
whereby sliding the protrusion into the bore to a first position causes the upper surface to be positioned above the lower socket wall so that the protrusion is free to rotate within the internal surface and further requiring a force to extract the protrusion from the internal surface; and
whereby sliding the protrusion into the bore to a second position causes the upper and lower surfaces to form discrete releasable connections with the upper and lower socket walls, respectively.

21. The attachment mechanism according to claim 20, wherein at least one of the releasable connections is a press fit.

22. The attachment mechanism according to claim 20, wherein the protrusion further includes a tapered surface located above the upper surface.

23. The attachment mechanism according to claim 20, wherein the internal surface further includes a tapered surface located below the lower socket wall.

24. An attachment mechanism for securely connecting components of a modular prosthesis, the attachment mechanism comprising:
a body comprising a top end, a bottom end, and an internal surface bounding a bore extending between the top end and bottom end; and
a stem having a protrusion, the protrusion having an external surface descending longitudinally downward from a free end, the protrusion being received within the bore of the body, the external surface of the stem biasing in frictional engagement directly against the internal surface of the body at a first location proximate to the top end of the body and at a second location proximate to the bottom of the body so that the body is held securely to the stem, a gap being formed between the internal surface of the body and the external surface of the stem along at least a portion of the distance between the first location and the second location.

25. The attachment mechanism of claim 24, wherein the frictional engagement is a press fit.

26. The attachment mechanism of claim 24, wherein the frictional engagement at one of the locations is a press fit and the frictional engagement at the other location is a self-locking taper.

27. The attachment mechanism of claim 24, wherein the external surface is contiguous.

28. The attachment mechanism of claim 24, wherein the gap has a length of at least 10 mm.

29. A method of assembling components of a modular prosthesis, comprising:
providing the attachment mechanism according to claim 1;
sliding the protrusion into the bore so that the lower surface is positioned below the lower socket wall and the upper surface is positioned above the lower socket wall;
rotationally orienting the stem relative to the body; and
sliding the protrusion further into the bore to create the simultaneous discrete releasable connections.
